(19) United States
(12) Patent Application Publication
(10) Pub. No.: US 2012/0277858 A1
(43) Pub. Date: Nov. 1, 2012

(54) IMPLANT FOR ANATOMICAL RECONSTRUCTION OR VOLUMETRIC AUGMENTATION

(76) Inventor: Thierry Brinon, Six-Fours-Les-Plages (FR)

(21) Appl. No.: 13/510,322

(22) PCT Filed: Nov. 10, 2010
(86) PCT No.: PCT/FR10/52410

§ 371 Date: Jun. 21, 2012

(30) Foreign Application Priority Data
Nov. 20, 2009 (FR) 0958210

(51) Int. Cl. A61F 2/12

(52) U.S. Cl. 623/8

(57) ABSTRACT

An implant for anatomically reconstructing, or increasing the positive displacement of, a soft portion of a living body is formed of a casing made of a flexible biocompatible material and filled with a gel. The inner surface of the cover that is in contact with the gel is texturized, comprising microscopic open cells.

### BACKGROUND

This invention relates to an implant for anatomical reconstruction or volumetric augmentation of a soft portion of a living body.

The main application of the invention is in the field of breast implants or prostheses suitable for being implanted under a person's skin, as a replacement or complement for the mammary gland, for anatomical and reparative construction after a mastectomy or ablation of said gland, or a volumetric augmentation for aesthetic purposes.

Other applications for other body areas are also possible with implants according to this invention, but the following description will mention and provide examples of only the most common human breast implants.

Such prostheses comprising an elastomeric envelope, preferably silicone, and silicone gel or physiological serum, which fills said envelope, are known: their shapes are round or anatomically symmetrical, or anatomically asymmetrical according to the desired effect.

Numerous patent applications have been filed in order to cover various manufacturing techniques, such as patent application FR 2 735 354, filed on Jun. 13, 1995 by the Perouse Implant Laboratory, which describes a breast prosthesis comprising a plurality of sacs filled with physiological serum and filling the external envelope in order to reduce the mobility of said fluid and obtain mechanical properties similar to those of a natural mammary gland.

Regardless of the shape or the technique for manufacturing such implants or prostheses, a first complication, which is now well known, has been observed in the post-operative period after implantation of the implant: capsular contracture. Indeed, the normal and constant physiological reaction of the human body in the presence of a foreign body is to isolate surrounding tissue by forming a hermetic membrane that will surround the implant and that is called a “peri-prosthetic capsule”. Normally, this membrane is fine, flexible and imperceptible, but it is possible for the reaction to be amplified and for the capsule to thicken, become fibrous and retract, compressing the implant, and is then referred to as a “shell”. Depending on the intensity of the phenomenon, the following may result: simple hardening of the breast, a sometimes irritating constriction, and even visible deformation with spheroidization of the prosthesis, resulting, in extreme cases, in a hard, painful and more or less off-centered sphere.

This retractile fibrosis, also called capsular contracture, is sometimes secondary to a hematoma or an infection, but usually its onset remains unpredictable, and results from random organic reactions.

Major progress has been made in recent years in this area, in surgical techniques and in particular in the design production of implants, resulting in a very substantial decrease in the rate of shells (or capsular contractures) and the intensity thereof, owing to implants with a textured external surface.

The rough external surface of the implant is generally obtained by using two types of manufacturing methods.

The first consists of spraying water-soluble crystals, generally sugar or salt, on the last layer soaked in the silicone dispersion. After catalysis of the envelope (baking), the envelopes are submerged in water in order to dissolve the crystals. After dissolution, the impression left on the envelope forms the rough surface thereof.

The particle size, shape and spraying force of the crystals define the final surface state of the envelope (roughness, depth and density of the pores). The patents FR 2 637 537 of Oct. 11, 1989 of the US CUI Corporation, patent application WO 2009/061672 of Oct. 31, 2008 of the Allergan company, or U.S. Pat. No. 5,674,285 filed on Dec. 12, 1995 by the Medical Products Development company, may be cited in order to illustrate this first type of method.

The other known manufacturing method consists of producing the rough surface on the mold of the envelope. The surface state of the mold may be obtained by blasting with a calibrated abrasive, by machining, by molding, or by any other technique enabling the surface state of the mold to be modified. In this case, it is the surface of the envelope in contact with the mold that becomes rough and, after mold release, the envelope is then turned over so as to obtain the rough external surface of the implant. To illustrate this second type of method, the patent EP 1 847 369 of Apr. 19, 2007 of the Cereplas company may be cited.

Thus, the breast implants currently known may consist of an envelope with a smooth or textured external surface.

A second post-operative complication is that of the diffusion of oil through the envelope and capable of causing the formation of siliconoma: indeed, silicone gel, which is the product often used to fill the envelope in order to give it its volume and provide the implant with the desired mechanical properties, is obtained by a mixture consisting primarily of silicone oil and a catalyst. The consistency of the gel is obtained after cross-linking (baking). Once cross-linked, by aging inside the envelope, the gel releases molecular chains referred to as short-chains at the surface, causing the oil to rise to the surface of the gel, and then through the envelope of the implant. This phenomenon, called transudation, is well known to a person skilled in the art.

Manufacturers of silicone and breast implants have developed solutions in order to make the implant envelopes less permeable and thus limit the migration of the short chains to the exterior of the implant: in this context, the patent application FR 2 498 446 of the Inserm company of Jan. 26, 1981 can be cited, which describes an implant with an hydrophilic external surface, which was initially developed in order to overcome the first above-mentioned complication of capsular contractures.

Another consequence of the rise of the oil to the surface of the gel, but which is less often described even though it is known, is the detachment of the gel from the envelope. This may cause deformation of the implant and the appearance of plications in the envelope. This may result in degradation of the aesthetic result of the implant, as well as a premature degradation of the envelope by shear force. This degradation is capable of going to the rupture of the envelope, which is then a third post-operative complication.

The rupture of breast implant envelopes is in fact the most common risk currently encountered with this type of implant, as the two complications described above may now be considered to have been overcome.

Indeed, even if the lifetime of a breast implant can be estimated at ten or even twelve years of implantation, a number of cases showed premature envelope ruptures well within these time periods.

The causes of these ruptures are numerous.

It is possible to distinguish two categories of implant ruptures:

ruptures independent of the quality of the implant,

ruptures directly linked to the quality of the implant.

Among the causes of breast implant envelope rupture independent of the quality of the implant, trauma associated with violent shock, alteration of the envelope during implantation (surgical instruments, suture needle) or even improper positioning of the implant causing folds in the envelope may be cited.

The quality of design and manufacture of the implant may also be a cause of premature rupture. Indeed, the choice of materials, the regularity of thickness of the envelope, and the mastery of the manufacturing method are also parameters that may have an impact on the mechanical strength of the envelope.

The silicone elastomers with which the prosthesis envelopes are generally produced are known for their excellent tolerance by the human body (biocompatibility) and their mechanical properties particular suitable for breast implants: flexibility, elasticity and shape memory (retentivity). Nevertheless, the resistance to tearing of these polymers remains their main drawback for this type of application.

Once an incipient rupture of a breast implant envelope appears, it develops and spreads as a result of stresses on the implant (movements, pressures, etc.). After several weeks or months, a rupture of several millimeters may reach several centimeters.

And, unlike breast implants pre-filled with physiological serum, of which the envelope rupture may immediately be diagnosed (the implant is emptied), a rupture of an implant pre-filled with silicone gel may be described as being “silent”. Indeed, insofar as the size of the opening does not enable the gel to migrate, the rupture is almost undetectable by palpation or by imaging means such as radiography, ultrasound and MRI.

It is when the gel has migrated out of the envelope that the rupture is generally diagnosed.

Indeed, for several years, manufacturers have developed silicone gels said to be highly cohesive in order to limit the risks of fragmentation of the gel in the event of an envelope rupture. However, this cohesiveness is entirely relative, and, in any case, the cohesiveness of the gel does not rule out the partial or total extraction of the gel from the envelope in the case of a large envelope tear.

The migration of the gel due to an implant rupture is, in all cases, a complication factor (pain, axillary ganglia) and requires surgical intervention (explantation).

During this intervention, after extraction of the ruptured implant, it is absolutely necessary to perform a curettage of the recess and the surrounding tissue in order to extract any trace of gel before implanting a new implant. However, the total extraction of gel fragments is particularly delicate and random, and it is common for siliconomas to reappear several months after the implant has been replaced.

### SUMMARY OF THE INVENTION

The objective of this invention is thus to prevent this third post-operative complication from occurring by providing an implant, in particular a breast implant, pre-filled with silicone gel, that does not carry the risk of deformation of the implant due to detachment of the gel from the envelope and that, in the event of rupture, regardless of the size of the rupture, ensures that the gel remains in the envelope.

This objective is achieved by an implant for anatomical reconstruction or volumetric augmentation of a soft portion of a living body, in which the implant consists of an envelope made of a flexible and biocompatible material filled with a gel, and the internal surface of the envelope in contact with the gel is, according to the invention, textured according to the direction and definition provided below, namely comprising small hollow areas, called cells, which are open, regular or not, and separated by areas called asperities, thus said to be in relief; the diameter and the depth of the cells, like the width and the height of the asperities, are several microns (μm): the microscopic dimensions of the cells and asperities give the internal surface of the envelope a fine roughness.

In a preferred embodiment of the invention, the envelope is made of elastomer and the gel is a silicone gel.

In the main application of the invention, the implant is a breast implant with a round, anatomically symmetrical or asymmetrical shape.

The result is a new implant of which the rough surface state of the interior of the envelope enables a surprisingly effective attachment of the gel to the envelope, which can be described as a cohesive bond.

Indeed, in various trials including that involving separation of the envelope with known implants that have smooth internal surfaces, it has been observed in order to illustrate the third post-operative complication described above that, by keeping the implant suspended by the apex of its dome fastened to a clamp (clothespin type), the weight of the implant applies a traction force that tends to detach the envelope from the gel: thus, by controlling the separation of the envelope at regular intervals, the results showed that the separation or detachment, which can be described as adhesive rupture, of the gel contained in the implant with the envelope having a smooth internal surface, is observed by the 10th day, and continues after the 30th day.

However, by taking an implant with an envelope having a textured internal surface having followed the same complete cycle of manufacturing, including sterilization, as the aforementioned implant with a smooth surface, and with samples having the same shape, same volume and manufactured with the same batch of filling gel and according to the same manufacturing conditions, no separation was observed with this implant of which the envelope has a textured internal surface, surprisingly, even after 100 days of suspension. Indeed, it may be considered that such a surface has a higher wettability with respect to a smooth surface or non-microscopic cells, and increases the mechanical adhesion between the gel and the envelope, but the absence of separation obtained is nevertheless surprising.

The new implant according to the invention satisfies the objective defined above, and the test results described above and those described below demonstrate the benefit: the description and the appended figures provide example embodiments, with the understanding that other embodiments are also possible in the context of the scope of this invention.

### BRIEF DESCRIPTION OF THE DRAWINGS

FIG. 1 is an example of a round breast implant from a perspective view.

FIG. 2A shows an implant according to the invention of which the envelope has been cut and remains secured to the gel in spite of the pressure exerted on the implant, and the gel is not extracted from the envelope.

FIG. 2B shows a known implant of which the envelope has a smooth internal surface and has been cut as with the implant of FIG. 2A: the pressure exerted on the implant separates the envelope from the gel, which leaves the prosthesis.

FIG. 3A shows a trial in which a test sample cut on the dome of an implant according to the invention is subjected to a traction force: there is no separation of the gel, and the latter breaks while remaining adhered to the test sample.

FIG. 3B shows the trial in which a test sample is subjected to a traction force as in FIG. 3A, cut on the dome of a known implant of which the envelope has a smooth interior: the test sample separates from the gel and no trace of it is visible on the test sample.

### DETAILED DESCRIPTION OF THE PREFERRED EMBODIMENT(S)

Any implant 1 intended for anatomical reconstruction or for volumetric augmentation of a soft portion of a living body, such as breast implants or prostheses, comprises of an envelope 3 made of a flexible and biocompatible material, preferably elastomer, and filled with a gel 2, preferably silicone.

The external surface 4₁ of the envelope 3 is smooth, textured or rough and, according to the invention, the internal surface 4₂ of the envelope 3 in contact with the gel 2 is textured, namely comprising small hollow areas, called cells, which are open, and regular or not, and separated by areas called asperities, thus said to be in relief; it may be considered that such a so-called textured internal surface is also rough.

To obtain the desired textured or rough effect, the depth and the diameter of the cells of the internal textured surface of the envelope 3 are microscopic, preferably between 5 and 40 μm and the density of these cells is preferably between 300 cells/cm² and 1800 cells/cm²; similarly, the asperities, which have the same microscopic height as the depth of the cells, also preferably have a microscopic width of between 5 and 40 μm; these cells are produced by any known method during the manufacture of said envelope 3.

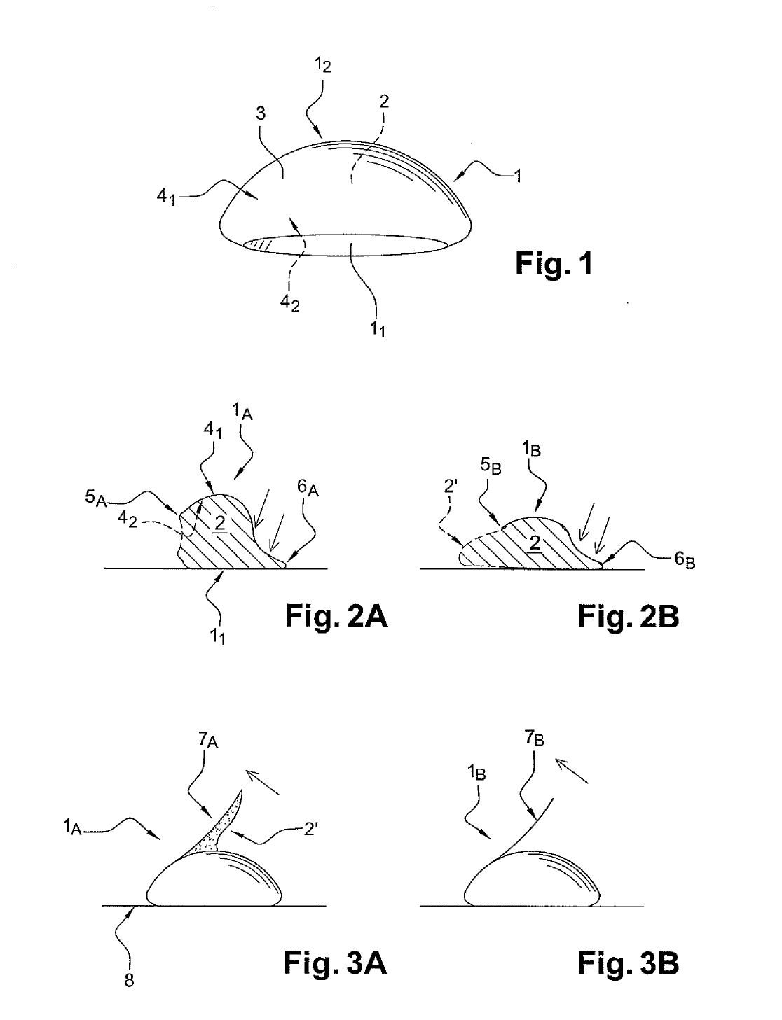

In the embodiment of FIG. 1, the breast implant 1 shown is round, with a portion 1₂ having a dome shape, intended to reproduce the anatomical shape of the breast to be reconstructed, or of which the volume is to be augmented, with a planar base 1₁.

The breast implant 1 May also have an anatomically symmetrical or asymmetrical shape.

FIGS. 2 and 3 show two trials complementary to that described above, and which confirm that the implants according to this invention solve the stated problem and that the implants currently known may cause the third post-operative complication described above in the event of a rupture of the envelope of the implant.

FIGS. 2 show comparative trials called migration trials, which involve producing an opening 5 in the implant 1 cut, for example with a scalpel and over a length corresponding to half the circumference of the implant shown in FIG. 1; the equator is the blend radius between the base 1₁ and the dome 1₂ of the implant. It is noted, as in the description of the first trial described above, that the implants la shown in FIGS. 2A and 3A according to the invention and those 1b shown in FIGS. 2B and 3B corresponding to the implants currently known, are samples having the same shape, the same volume and manufactured with the same filling gel batch and according to the same manufacturing conditions with an identical complete cycle, including sterilization.

In this second trial, the samples are then kept suspended by a clamp fastened opposite 6a the opening 5a, respectively 6b and 5b, and every twenty-four hours, each implant is subjected to five sequential pressures, by placing, according to FIGS. 2, these implants on a planar surface 8 and by pressing on the dome 1₂ so as to reproduce the stresses to which a prosthesis implanted in a human body and of which the envelope is ruptured might be subjected.

At regular intervals, the separation and flow of the gel 2 from the envelope 3 are checked.

Such trials have thus shown that the gel 2, contained in the envelope 3, having a smooth internal surface, of the implants currently known, is gradually detached from said envelope 3 and is gradually extracted 2′ from said envelope after 40 days of suspension. After 130 days of suspension, the gel 2 is entirely extracted 2′ from the envelope 3.

By contrast, the implant la according to FIG. 2A, of which the envelope 3 has an internal textured surface according to the invention, does not show any separation or extrusion of the gel 2 from the envelope, even after 280 days of suspension.

The series of third trials shown in FIGS. 3 consists of measuring, with a dynamometer, the traction force necessary for separation of the envelope 2 and of analyzing the internal surface thereof at the end of the trial. For this, a rectangular test sample with sides of 100 mm and 15 mm, centered and symmetrically distributed at the apex of the dome 1₂ of each implant 1_{A} and 1_{B}, is cut and left in place.

One of the two ends of the test sample 7 is clamped in the mobile jaw of a dynamometer and the base of the implant is held on the stationary plate 8 of the dynamometer. The test samples 7_{A} and 7_{B} are subjected to a traction force at a rate of 20 mm per minute until the test sample considered, in contact with the gel 2, is entirely separated.

The trial of FIG. 3B, in which the implant 1_{B} has a smooth internal surface, showed that the test sample 7_{B} was separated with a force of less than 0.5 Newton and that, at the end of the trial, the smooth internal surface of the test sample 7_{B} did not have any trace of gel.

Conversely, in FIG. 3A with an implant according to the invention having a textured internal surface, the test sample 7_{A} was detached from the gel 2 with a force of 0.7 Newton and was entirely covered with gel 2′ at the end of the trial. In this case, the gel was broken.

As described above, the texturization of the envelope 3 May be obtained:

by spraying soluble crystals on the surface of the non-cross-linked elastomer, then by dissolving the crystals after cross-linking of the elastomer,

or by modifying the surface state of the mold on which the elastomer envelope is produced.

In the case of the embodiment of the breast implant of the invention in which the external surface 4₁ of the envelope 3 is smooth, the internal texture 4₂ of the envelope may be obtained by either of the two methods described above.

In the case of the embodiment of the breast implant of the invention in which the external surface 4₁ is textured, both methods are then necessary: the texture of the internal face on the mold and the texture of the external surface by spraying soluble crystals, or the reverse.

1-9. (canceled)

10. Implant for anatomical reconstruction or for volumetric augmentation of a soft portion of a living body, comprising an envelope made of a flexible and biocompatible material and filled with a gel, wherein an internal surface of the envelope in contact with the gel is textured, comprising open cells of microscopic dimensions.

11. Implant according to claim 10, wherein the envelope is made of elastomer.

12. Implant according to claim 10, wherein the gel is a silicone gel.

13. Implant according to claim 10, wherein an external surface of the envelope is smooth.

14. Implant according to claim 10, wherein an external surface of the envelope is textured.

15. Implant according to claim 10, wherein the implant is a round breast implant.

16. Implant according to claim 10, wherein the implant is a breast implant with an anatomically symmetrical or asymmetrical shape.

17. Implant according to claim 10, wherein a depth of the cells of the internal textured surface of the envelope is between 5 and 40 μm (microns).

18. Implant according to claim 10, wherein a density of said cells of the internal textured surface of the envelope is between 300 cells/cm² and 1800 cells/cm².
